(19) United States
(12) Patent Application Publication
(10) Pub. No.: US 2024/0390592 A1
(43) Pub. Date: Nov. 28, 2024

(54) Controlling a sensor of a drug delivery device or of a drug delivery add-on device

(71) Applicant: SANOFI, Paris (FR)

(72) Inventors: Jonathan Jason Drake, Warwick (GB); Ronald Antony Smith, Warwick (GB)

(21) Appl. No.: 18/694,272

(22) PCT Filed: Sep. 22, 2022
(86) PCT No.: PCT/EP2022/076291

§ 371 Date: Mar. 21, 2024

(30) Foreign Application Priority Data
Sep. 24, 2021 (EP) 21315172.3

(51) Int. Cl. A61M 5/315; A61M 5/24; A61M 5/31; A61M 5/32

(52) U.S. Cl. CPC A61M 5/31556; A61M 5/24; A61M 5/31528; A61M 5/31551; A61M 5/3202; A61M 2005/3126; A61M 2202/0007; A61M 2202/04; A61M 2205/3313; A61M 2205/3327; A61M 2205/50; A61M 2205/8206

(57) ABSTRACT

A method for controlling a sensor of a drug delivery device or of a drug delivery add-on device is disclosed, wherein the sensor comprises a light emitter and a phototransistor as light receiver, and wherein the method comprises generating a first drive signal for the light emitter and a second drive signal for the phototransistor, wherein the second drive signal is generated to bias the phototransistor if the first drive signal is generated to switch off the light emitter, and the second drive signal is generated to acquire an output signal of the phototransistor if the first drive signal generated to switch on the light emitter.

### CROSS REFERENCE TO RELATED APPLICATIONS

The present application is the national stage entry of International Patent Application No. PCT/EP2022/076291, filed on Sep. 22, 2022, and claims priority to Application No. EP 21315172.3, filed on Sep. 24, 2021, the disclosures of which are incorporated herein by reference.

### TECHNICAL FIELD

The present disclosure relates to controlling a sensor of a drug delivery device or of a drug delivery add-on device.

### BACKGROUND

WO2016131713A1 relates to a data collection device for attachment to an injection device and collecting medicament dosage information therefrom. The data collection device may comprise a mating arrangement configured for attachment to the injection device, a sensor arrangement configured to detect movement of a movable dosage programming component of the injection device relative to the data collection device during delivery of a medicament, and a processor arrangement configured to, based on said detected movement, determine a medicament dosage administered by the injection device. The sensor arrangement may include an optical sensor, for example, an optical encoder unit, particularly including a light source, such as a light emitting diode (LED) and a light detector, such as an optical transducer. The processor arrangement may be configured to monitor a time period elapsed since a pulse was output by the optical encoder and to determine said medicament dosage if said time period exceeds a predetermined threshold.

WO2019101962A1 relates to medicament injection devices. An injection device May comprise a movable dosage programming component comprising a rotary encoder system having a predefined angular periodicity, a sensor arrangement comprising a first optical sensor configured to detect movement of the movable dosage programming component relative to the sensor arrangement during dosing of a medicament, wherein the first optical sensor is configured to operate in a strobe-sampling mode at a first frequency, and a second optical sensor configured to detect movement of the rotary encoder system relative to the second optical sensor, wherein the second optical sensor is configured to operate in a strobe-sampling mode at a second frequency lower than the first frequency, and a processor arrangement configured to, based on said detected movement, determine a medicament dosage administered by the injection device. A controller may be provided to control a sensor arrangement comprising the optical sensors, for example infrared (IR) reflective sensors, which emit IR light from an LED and detect IR light reflected from IR reflective regions of the encoder system.

### SUMMARY

This disclosure describes methods and devices for controlling a sensor of a drug delivery device or of a drug delivery add-on device.

In one aspect the present disclosure provides a method for controlling a sensor of a drug delivery device or of a drug delivery add-on device, wherein the sensor comprises a light emitter and a phototransistor as light receiver, wherein the method comprises generating a first drive signal for the light emitter and a second drive signal for the phototransistor, wherein the second drive signal is generated to bias the phototransistor if the first drive signal is generated to switch off the light emitter, and the second drive signal is generated to acquire an output signal of the phototransistor if the first drive signal generated to switch on the light emitter. By biasing the phototransistor and driving the IR LED appropriately the response time of the sensor can be significantly improved compared to the manufacturer's specifications. The method can be applied to any application of a sensor of a drug delivery device or drug delivery add-on device having a phototransistor as light receiver. Drug delivery devices may e.g. be injection pens or autoinjectors. The method is particularly suitable for application with sensors used in drug injection pens employing optical encoder systems to detect drug dosage selection and expelling, for example an injection pen as described in WO2014033195, particularly to improve the accuracy of dosage measurements with the optical encoder system. The term “light” as used herein is to be understood to comprise electromagnetic radiation within a portion of the electromagnetic spectrum, which comprises the visible light perceivable by the human eye as well as infrared (IR) light and ultraviolet (UV) light.

In an embodiment, the second drive signal may be generated by default to bias the phototransistor. Thus, the phototransistor may be immediately upon powering a measurement electronics of a drug delivery device operated in a mode, in which measurements can be immediately taken (e.g., according to a response speed of the phototransistor). The measurement accuracy may be obtained nearly immediately upon powering the drug delivery or drug delivery add-on device.

In embodiments, generating the first drive signal to switch on the light emitter may comprise generating an electric current pulse of a predefined pulse time, which is selected such that the output signal of the phototransistor may reach a predefined value, particularly about 66% of the full scale of an analogue-to-digital converter. This allows to obtain good signal levels of the output signal of the phototransistor, well away from saturation, with headroom for tolerances, and a high dynamic range whilst using minimal power for emitting light with the light emitter.

In a further embodiment, the second drive signal to acquire an output signal of the phototransistor may be generated nearly at the same time of generating the first drive signal to switch on the light emitter or a predefined time before the first drive signal to switch on the light emitter or a predefined time delay after generating the first drive signal to switch on the light emitter. A synchronous generation of the first and second drive signals, i.e. at nearly the same time, may be normal, but also a delayed generation of the second drive signal after the generation of the first drive signal can be employed, particularly if the response of the phototransistor to a received light pulse may be delayed.

In yet further embodiments, generating the second drive signal to acquire an output signal of the phototransistor may comprise generating a switching signal to connect a signal acquisition input with an output of the phototransistor to receive the output signal of the phototransistor for a predefined acquisition time, particularly being longer than the predefined pulse time. The switching signal may for example control a switch, for example a transistor, to switch between a voltage potential for biasing the phototransistor and a signal acquisition circuitry, for example a sample and hold circuit.

In embodiments, the second drive signal may be generated to bias the phototransistor by pulling an output of the phototransistor to a predefined voltage potential, particularly 0 volts. Thus, no specific voltage source needs to be provided, but a ground connection can be sufficient.

In embodiments, the method may comprise acquiring the output signal of the phototransistor by sampling and converting the output signal into a digital signal. A digital signal may be directly processed digitally with logic circuitry, particularly a processor.

In a further aspect the present disclosure provides a device for controlling a sensor of a drug delivery device or of a drug delivery add-on device, wherein the sensor comprises a light emitter and a phototransistor as light receiver, wherein the device is configured to implement a method as disclosed herein. The device may be implemented for example by an electronic circuitry, such as an ASIC (Application Specific Circuit), a (F) PGA ((Field) Programmable Gate Array), Programmable Logic Device (PLD) or a processor, including but not limited to microcontroller, Digital Signal Processor (DSP), Floating Point Unit (FPU), sensor controller, or motion controller.

In an embodiment, the device may comprise a controller, particularly a microcontroller, being configured to generate the first drive signal for the light emitter and the second drive signal for the phototransistor and comprising an input for connecting to an output of the phototransistor to receive the output signal of the phototransistor, wherein the input can be controller-internally switched between a predefined voltage potential, particularly 0 volts, an input of an analogue-to-digital converter comprised by the controller. A controller may comprise in addition to a processor further dedicated circuitry, such as voltage and current sources for biasing the phototransistor, control circuitry for generating a control signal for a light emitter, and circuitry for acquiring the output signal of the phototransistor, and it can be programmed with a dedicated firmware to perform the method as disclosed herein.

In a further embodiment, the input of the analogue-to-digital converter may comprise an input capacitance for receiving an electric charge from the phototransistor and selected to obtain a rising time of an input voltage of the analogue-to-digital converter being lower than a predefined rising time.

In a still further aspect the present disclosure provides a sensor for a drug delivery device or for a drug delivery add-on device, wherein the sensor comprises at least one sensor unit comprising a light emitter configured to emit light in a first frequency range and a phototransistor configured to detect received light in a second frequency range, wherein the second frequency range comprises the first frequency range, and a device as disclosed herein and configured to control the at least one sensor unit.

In an embodiment, the light emitted in the first frequency range by the light emitter may comprise a first peak wavelength and a first full-width half-maximum and the phototransistor light detection spectrum comprise a second peak wavelength and a second full-width half-maximum, wherein the first peak wavelength and the first full-width half-maximum are selected such that the spectrum of the emitted light is comprised by the phototransistor light detection spectrum and light emitted from the light emitter and received by the phototransistor generates a signal level of the output signal of the phototransistor sufficient for further processing by the device. Thus, the phototransistor is adequate to ensure detection of light emitted by the light emitter. Particularly, the phototransistor may have a basis that provides a reasonable signal level for a given emitter drive current and target reflectance.

In a further embodiment, the first peak wavelength may be about 936 nm and the first full-width half-maximum is about 59 nm, and wherein the second peak wavelength is about 872 nm and the second full-width half-maximum 276 nm. A light emitter with such a first peak wavelength may have a very fast response time relative to a coupled phototransistor with the second peak wavelength.

In further embodiments, phototransistor may be connected to a load of about 47 kΩ (kilo Ohm). It has been discovered that by using suitable circuitry the effective rise time of the output signal of phototransistor with such a load can be made faster. In specific embodiments of the phototransistor circuitry, the effective rise time may be reduced to about 3.0 us with a 47 kΩ load, and also the fall time may be much shorter when the load resistance may be actively lowered to less than 100 Ohm.

In a yet further aspect the present disclosure provides a drug delivery device or a drug delivery add-on device, particularly an injection pen, comprising a body for holding a drug container, a dosage selection mechanism for selecting a drug dosage to be delivered, which comprises an optical encoder system for detecting a selected and/or delivered drug dosage, and a sensor as disclosed herein and arranged to detect movement of a part of the optical encoder system upon drug dosage selection and/or delivery based on the detection of reflections of emitted light from the moving part of the optical encoder with the phototransistor.

### BRIEF DESCRIPTION OF THE FIGURES

FIG. 1 shows an injection device according to a first embodiment;

FIG. 2 is an elevated side view of a first type of encoder system;

FIG. 3 is a plan view of the encoder system shown in FIG. 2;

FIG. 4 shows a schematic block diagram of an embodiment of a device controller;

FIG. 5 shows a datasheet of photo-reflective sensor with manufacturer's test definitions and response times as a function of load resistance;

FIG. 6 shows a circuit diagram of an embodiment of a device for controlling a photo-reflective sensor;

FIG. 7 shows simplified functional waveforms of signals of the device of FIG. 6.

### DETAILED DESCRIPTION

In the following, embodiments of the present disclosure will be described with reference to injection devices, particularly an injection device in the form of a pen. The present disclosure is however not limited to such application and may equally well be deployed with other types of drug delivery devices, particularly with another shape than a pen. All absolute values are herein shown by way of example only and should not be construed as limiting.

An example of an injection pen including an injection button and a grip that are combined is described in WO2014033195. Another example of an injection device where there are separate injection button and dial grip components is described in WO2004078239.

In the following discussion, the terms “distal”, “distally” and “distal end” refer to the end of an injection pen towards which a needle is provided. The terms “proximal”, “proximally” and “proximal end” refer to the opposite end of the injection device towards which an injection button or dosage knob is provided.

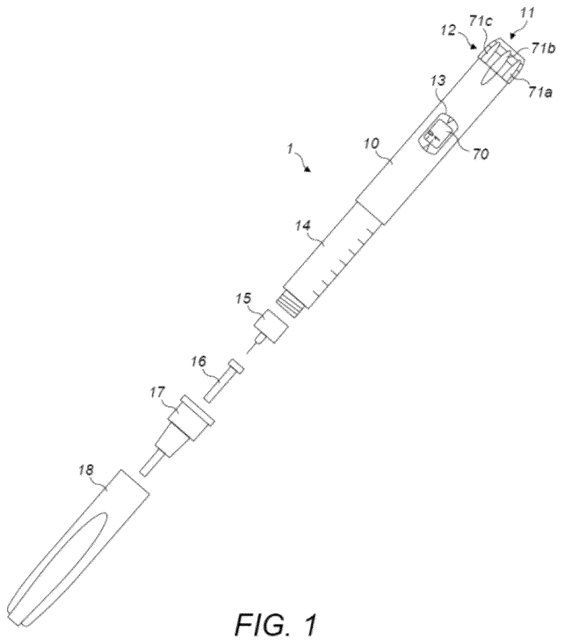

FIG. 1 is an exploded view of an injection pen 1, such as described in WO2014033195. The injection pen 1 of FIG. 1 is a pre-filled, disposable injection pen that comprises a housing 10 and contains an insulin container 14, to which a needle 15 can be affixed. The needle is protected by an inner needle cap 16 and either an outer needle cap 17 other cap 18. An insulin dose to be ejected from injection pen 1 can be programmed, or ‘dialled in’ by turning a dosage knob 12, and a currently programmed dose is then displayed via dosage window 13, for instance in multiples of units. For example, where the injection pen 1 is configured to administer human insulin, the dosage may be displayed in so-called International Units (IU), wherein one IU is the biological equivalent of about 45.5 micrograms of pure crystalline insulin (1/22 mg). Other units may be employed in injection devices for delivering analogue insulin or other medicaments. It should be noted that the selected dose may equally well be displayed differently than as shown in the dosage window 13 in FIG. 1.

The dosage window 13 may be in the form of an aperture in the housing 10, which permits a user to view a limited portion of a dial sleeve 70 that is configured to move when the dosage knob 12 is turned, to provide a visual indication of a currently programmed dose. The dosage knob 12 is rotated on a helical path with respect to the housing 10 when turned during programming. In this example, the dosage knob 12 includes one or more formations 71a, 71b, 71c to facilitate attachment of a data collection device (drug delivery or injection add-on device).

The injection pen 1 may be configured so that turning the dosage knob 12 causes a mechanical click sound to provide acoustical feedback to a user. The dial sleeve 70 mechanically inter-acts with a piston in insulin container 14. In this embodiment, the dosage knob 12 also acts as an injection button. When needle 15 is stuck into a skin portion of a patient, and then dosage knob 12 is pushed in an axial direction, the insulin dose displayed in display window 13 will be ejected from injection pen 1. When the needle 15 of injection pen 1 remains for a certain time in the skin portion after the dosage knob 12 is pushed, a high percentage of the dose is actually injected into the patient's body. Ejection of the insulin dose may also cause a mechanical click sound, which is however different from the sounds produced when rotating the dosage knob 12 during dialling of the dose.

In this embodiment, during delivery of the insulin dose, the dosage knob 12 is returned to its initial position in an axial movement, without rotation, while the dial sleeve 70 is rotated to return to its initial position, e.g. to display a dose of zero units.

Injection pen 1 may be used for several injection processes until either the insulin container 14 is empty or the expiration date of the medicament in the injection pen 1 (e.g. 28 days after the first use) is reached.

Furthermore, before using injection pen 1 for the first time, it may be necessary to perform a so-called “prime shot” to remove air from insulin container 14 and needle 15, for instance by selecting two units of insulin and pressing dosage knob 12 while holding injection pen 1 with the needle 15 upwards. For simplicity of presentation, in the following, it will be assumed that the ejected amounts substantially correspond to the injected doses, so that, for instance the amount of medicament ejected from the injection pen 1 is equal to the dose received by the user. Nevertheless, differences 30 (e.g. losses) between the ejected amounts and the injected doses may need to be taken into account.

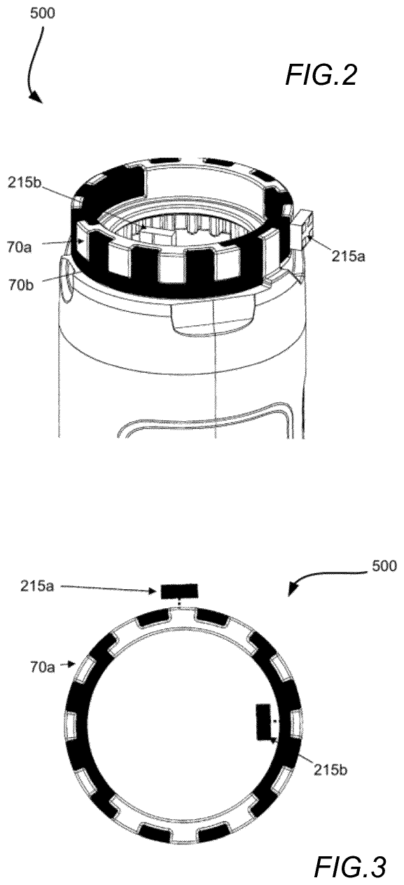

As explained above, the dosage knob 12 also functions as an injection button so that the same component is used for dialling and dispensing. A sensor arrangement 215 (FIGS. 2 and 3) comprising one or more optical sensors may be mounted in the injection button or dosage knob 12 which is configured to sense the relative rotational position of the dial sleeve 70 relative to the injection button 12. This relative rotation can be equated to the size of the dose dispensed and used for the purpose of generating and storing or displaying dose history information. The sensor arrangement 215 may comprise a primary (optical) sensor 215a and a secondary (optical) sensor 215b. The sensor arrangement 215 may be also mounted in drug delivery or injection add-on device, which may be provided for usage with different injection devices 1 and configured to collect data acquired with the sensor arrangement 215.

The optical sensors 215a, 215b of the sensor arrangement 215 may be employed with an encoder system, such as the system 500 shown in FIGS. 2 and 3. The encoder system is configured for use with the device 1 described above. As shown in FIG. 2 and FIG. 3, the primary sensor 215a and secondary sensor 215b are configured to target specially adapted regions at the proximal end of the dial sleeve 70. In this embodiment, the primary sensor 215a and secondary sensor 215b are IR reflective sensors. Therefore, the specially adapted proximal regions of the dial sleeve 70 are divided into a reflective area 70a and a non-reflective (or absorbent) area 70b. The part of the dial sleeve 70 comprising the reflective area 70a and a non-reflective (or absorbent) area 70b may be termed an encoder ring.

To keep production costs to a minimum, it may be favourable to form these areas 70a, 70b from injection moulded polymer. In the case of polymer materials, the absorbency and reflectivity could be controlled with additives, for example carbon black for absorbency and titanium oxide for reflectivity. Alternative implementations are possible whereby the absorbent regions are moulded polymer material and the reflective regions are made from metal (either an additional metal component, or selective metallisation of segments of the polymer dial sleeve 70).

It is also possible to use variation in detecting range, or to have a reflector and absence of reflector/gap. Gradation of reflectivity and/or absorbance could also be used.

The second detector may be rotationally offset by half the width of the first detector flags in order to detect the direction of rotation.

It can be noted that the embodiment shown in FIGS. 2 and 3 with the optical sensors 215a, 215b of the sensor arrangement 215 being arranged in the same housing is an implementation, which allows a close spatial neighbourhood between emitter and receiver, which may be advantageous in some implementations, particularly due to the short time of flight. Generally, it may be advantageous when emitter and receiver are arranged close together also with regard to the reflective area 70a. However, it can be further noted that emitter and receiver may also be arranged in different housings or parts of the housing, or they may be spatially separated from each other without deviating from the spirit of this disclosure. For example, they could even be part of different circuit boards and microcontrollers that are interconnected with each other,

Having two sensors facilitates a power management technique described below. The primary sensor 215a is arranged to target a series of alternating reflective regions 70a and non-reflective regions 70b at a frequency commensurate with the resolution required for the dose history requirements applicable to a particular drug or dosing regime, for example, 1 IU. The secondary sensor 215b is arranged to target a series of alternating reflective regions 70a and non-reflective regions 70b at a reduced frequency compared to the primary sensor 215a. It can be understood that the encoder system 500 could function with only a primary sensor 215a to measure the dispensed dose. The secondary sensor 215b facilitates the power management technique described below.

The two sets of encoded regions 70a, 70b are shown in FIGS. 2 and 3 concentrically with one external and the other internal. However, any suitable arrangement of the two encoded regions 70a, 70b is possible. Whilst the regions 70a, 70b are shown as castellated regions, other shapes and configurations are possible.

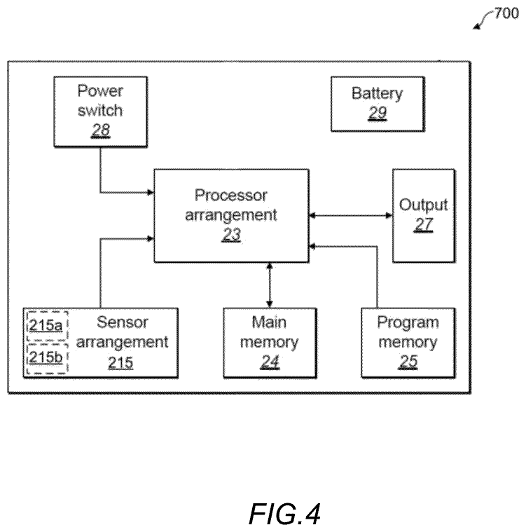

The device 1 or an add-on device for attachment to the device 1 may also include a controller 700, as shown schematically in FIG. 4. The controller 700 comprises a processor arrangement 23 including one or more processors, such as a microprocessor, a Digital Signal Processor (DSP), Application Specific Integrated Circuit (ASIC), Field Programmable Gate Array (FPGA) or the like, together with memory units 24, 25, including program memory 24 and main memory 25, which can store software for execution by the processor arrangement 23. The controller 700 is provided and configured to control the sensor arrangement 215. An output 27 is provided, which may be a wireless communications interface for communicating with another device via a wireless network such as Wi-Fi™ or Bluetooth®, or an interface for a wired communications link, such as a socket for receiving a Universal Serial Bus (USB), mini-USB or micro-USB connector. For example, data may be output to a data collection device attached to the device 1. A power switch 28 is also provided, together with a battery 29. The power switch 28 may comprise the function of a system state switch for toggling the system state in embodiments with an always present power, i.e., where the power is never switched off.

It is advantageous to be able to minimise the power usage of the encoder system 500 so that the size of a battery 29 needed to be packaged into the device 1 can be minimised. The sensors 215a, 215b used in this embodiment require a certain amount of power to operate. This embodiment is arranged such that the sensors 215a, 215b can be switched on and off intermittently at a controlled frequency (i.e. in a strobe-sampling mode). There is inherently a limit to the maximum rotational speed that can be counted by a sampled encoder system before aliasing occurs. Aliasing is the phenomenon where the sampling rate is less than the rate at which sensed regions pass the sensor which means that a miscount could occur when a region change is missed. The secondary sensor 215b with a reduced frequency compared to the primary frequency 215a can tolerate a higher rotational speed before it too becomes aliased. Whilst the secondary sensor 215b is not able to resolve the dose dispensed to the same resolution as the primary sensor 215a, the output of the secondary sensor 215b remains reliable at higher speeds. Therefore both sensors 215a, 215b are used in combination to be able to accurately determine dose delivered up to a first threshold rotational (dispensing) speed. The sensors 215a, 215b can then be used to determine an approximate dose delivered up to a second (higher) threshold dosing speed. At speeds above the second threshold speed the sensors 215a, 215b will not be able to accurately or approximately determine the dose delivered, therefore the second threshold is set above a speed which is not physically possible in the injection pen 1.

The first speed threshold is determined by the sampling rate of primary sensor 215a and the frequency of encoder region transitions, which is fixed at the resolution required by the intended drug or dosing regimen (for example one transition per 1 IU). The second speed threshold is determined by the sampling rate of the secondary sensor 215b and the frequency of encoder region transitions. The first threshold is set such that the largest range of dispensing speeds can be covered by the system for accurate reporting of dose dispensed.

The example embodiment shown in FIG. 3 has primary sensor 215a targeting region transitions at 1 transition per 1 IU of dose delivered and the secondary sensor 215b targeting region transitions at 1 transition per 6 IU of dose delivered. Other options are possible which include 1 transition per 2 IU, 1 transition per 4 IU, 1 transition per 8 IU and 1 transition per 12 IU units. These options are each possible because there are 24 separate regions 70a, 70b per revolution in the encoder system 500 shown in FIG. 3. In general, if the number of separate regions 70a, 70b per revolution were n units then there would be options at one region transition per m units where m was any integer factor of n greater than 1 and less than n.

The slower the sampling frequency of both sensors 215a, 215b, the lower the power consumption required and therefore the smaller the required size of the battery 29. It is therefore optimal to minimise, by design, the sampling frequency as far as is practical.

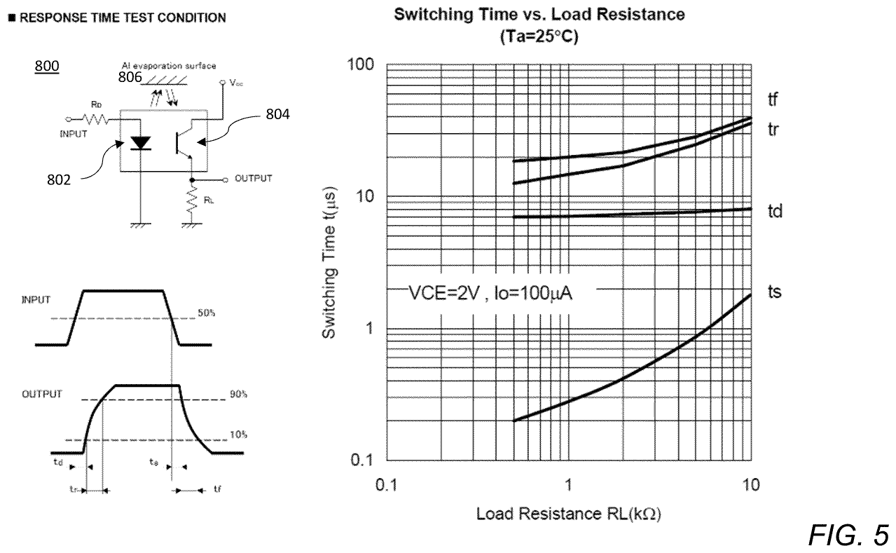

FIG. 5 shows an example an optical sensor package 800, which can be used to implement a sensor 215a, 215b of the sensor arrangement 215, and the typical course of the input and output signal of this optical sensor as well as its switching time depending on the load resistance. The sensor package is a photo-reflective sensor. It comprises two elements, an IR LED (emitter) 802 and an IR sensitive phototransistor (receiver) 804. The two elements are arranged such that light from the emitter 802 will be directed onto the receiver 804 when incident upon a target 806 (such as the reflective regions 70a of the encoder system shown in FIGS. 2 and 3) that is suitably oriented and with some reflectivity. The received reflected light is converted to an electric charge within the receiver (phototransistor) 804 and output as a signal that represents that quantity of light. With some prior knowledge of the target, it is possible to infer characteristics of the target from the detected light.

The emitter 802 within the sensor package is an IR LED may comprise a peak wavelength of about 936 nm and full-width half-maximum (FWHM) of 5.2% (59 nm). It may have a very fast response time relative to the coupled receiver 804. With a much broader spectral response than the emitter 802, centred at 872 nm with FWHM of 31.6% (276 nm), the receiver 804 may be adequate to ensure detection of light from the IR LED. It has a phototransistor basis that provides a reasonable signal level for a given emitter drive current and target reflectance. Typical input and output signal responses with rise and fall times are shown below the circuit diagram of the sensor package.

The phototransistor response time may be largely dominated by two mechanisms, transit of charge carriers across the depletion region (fast) and diffusion of carriers (slow); thus, the response time may have two components. The combined response times, as usually presented by manufacturers of such sensor packages, are dominated by the slow mechanism. Further, the definition of the rise time (10% to 90%) may tend to bias the figures towards longer response times. A typical datasheet of such a sensor package may quote a typical rise time (tr) of 35 us and fall time (tf) of 40 us with a 10 kΩ load RL. It has been discovered that by using suitable circuitry at the output side of the package, the effective rise time can be reduced to about 3.0 us with a 47 kΩ load RL. The fall time can be also reduced when the load resistance is actively lowered to less than 100 Ohm by suitable circuitry at the output side.

An exemplary characteristic of a sensor package is shown in the right diagram of FIG. 5, which shows the switching time t in us depending on the load resistance RL in kΩ with a voltage VCE of 2 Volts at the output of the phototransistor and an output current lo of 100 μA under an operating temperature Ta of about 25° C.

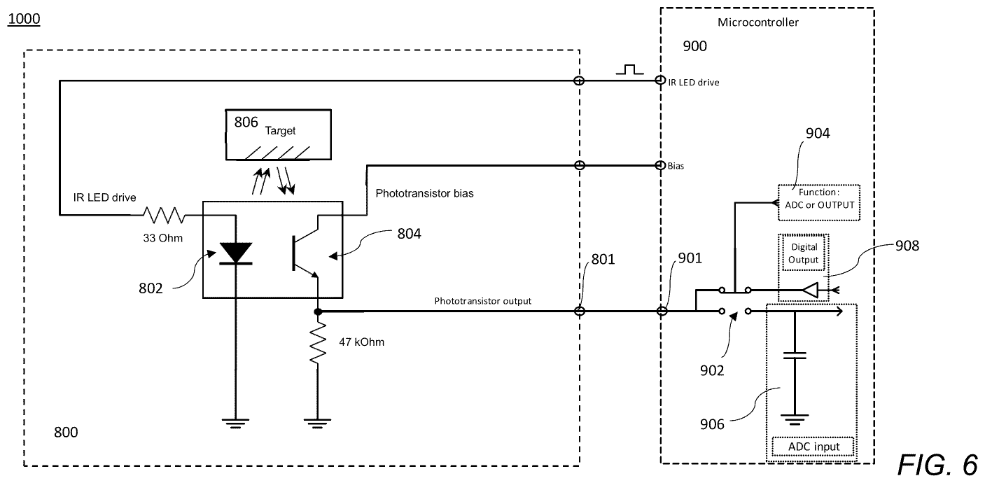

The basic sensor operation will now be described with reference to FIG. 6, which shows a circuit diagram of an embodiment of a device 1000 for controlling a photo-reflective sensor. The device 1000 comprises the sensor package 800 and the controller 900 being configured for generating drive signals for the sensor package 800 (output “IR LED drive” for driving the light emitter 802 and output “Bias” for driving the phototransistor 804) and for processing the output signal at the “Phototransistor output” of the sensor package 800.

The circuitry of the sensor package 800 comprises a 33 Ohm resistance between the “IR LED drive” output of the controller 900 and an input of the light emitter, the IR LED 802, particularly to limit the electric current through the IR LED 802, and a 47 kΩ load resistance between ground potential and the emitter of the phototransistor 804. The collector of the phototransistor 804 is connected to the “Bias” output of the controller 900.

The controller 900 comprises an internal switch 902, which is provided to either connect the input 901 of the controller 900 to a controller-internal analogue-to-digital converter (ADC) input 906 or to a controller-internal digital drive 908. The input 901 may be a multipurpose pin of the controller 900 being configurable as input or output. The switch 902 is controlled by a switch function 904, which may be for example implemented by a firmware function of the controller 900 or a dedicated logic circuitry of the controller 900. The controller 900 may be for example comprised by the processor arrangement 23 of FIG. 4.

The device 1000 may be implemented by means of a PCB (Printed Circuit Board), particularly a flexible PCB for integration into the injection device 1 from FIG. 1, on which the sensor package 800 as single chip and the controller 700 are soldered. Also other implementations are possible, for example a PCB comprising only the controller 700 and some other electronic device and a wiring connecting the controller pins with the respective pins of the separate sensor package 800, which may for example be integrated in the dosage mechanism of the injection device 1. A further possible embodiment may comprise a drug injection add-on device, which may comprise the controller 900, while the sensor package 800 is contained in the injection device.

Connectors may be provided to make electrical connections between the respective connectors of the sensor package 800 and the controller 900 when the add-on device is attached to the drug injection device. A yet further possible implementation may comprise a single device solution, for example a SoC (System on Chip) with the controller 900 integrated with the sensor package 800 into a single device.

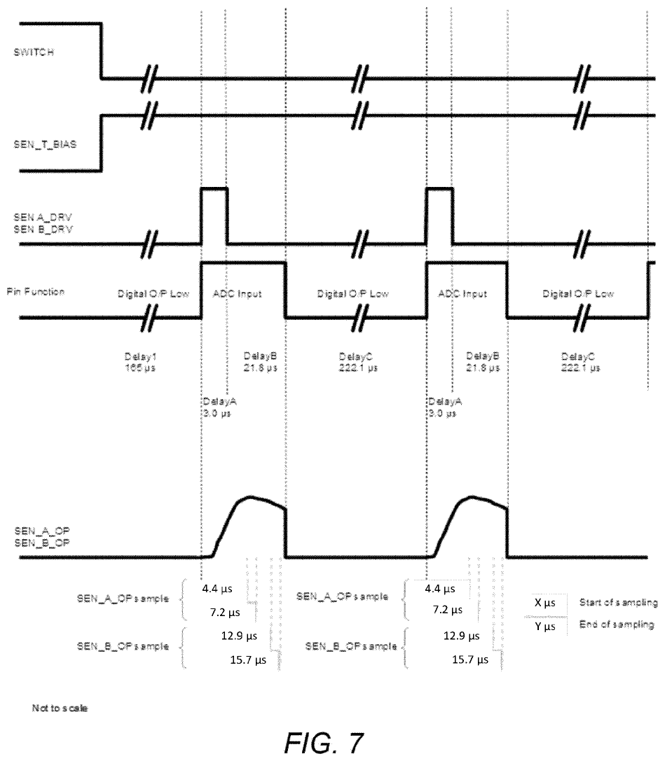

Next, several aspects of the operation and implementation of the device 900 will be described with reference to FIGS. 6 and 7.

Basic sample function: on activation of the device 900, the controller 900 generates a bias as driver signal, which is applied via the respective connection to the collector of the phototransistor 804. The bias signal for the phototransistor 804 puts the collector at a suitable bias voltage (signal “SEN_T_BIAS” in FIG. 7). This may be one of the first actions of the controller 900. The switch control function 904 of the controller 900 further configures the pin 901 as output by connecting it via the switch 902 with the digital output 908 (signal “Pin Function” in FIG. 7). Most of the time the pin 901 is configured as an output at the system reference potential, particularly designated 0V or ground. This applies the full bias voltage across the phototransistor 804 for a significant time in order to stabilise the sensor package 800 prior to the first sample. When a sample is to be taken, the controller 900 transiently converts the pin 901 from output or digital drive to the ADC input 906 by controlling the switch 902 to disconnect the pin 901 from the digital drive 908 and to connect it with the ADC input 906 (signal “Pin Function” in FIG. 7). The controller also drives the sensor's IR LED 802 by generating an IR LED drive signal, for example a drive pulse (signal “SEN_A_DRV” for a first one of two sensors, for example sensors 215a, 215b from FIGS. 2 and 3, and signal “SEN_B_DRV” for a second one of two sensors in FIG. 7). The IR LED 802 emits a light and the biased phototransistor 804 receives the reflections of the emitted light from the target 806, thus taking a reading or a sample (signal “SEN_A_OP” for a first one of two sensors, for example sensors 215a, 215b from FIGS. 2 and 3, and signal “SEN_B_OP” for a second one of two sensors in FIG. 7). As soon as the reading is complete, the input is converted to an output again by the switch control function 904, which disconnects pin 901 from the ADC input 806 and connects it with the digital drive 908 (signal “Pin Function” in FIG. 7). Since the bias is constantly applied, the period between samples is used to condition the phototransistor 804 prior to the next reading. Any residual charge within the phototransistor 804 is swept as fast as possible under the electric field provided by the bias. The bias is held until no further samples are to be taken then it is set to the same voltage as the phototransistor output 801 thereby shorting the phototransistor 804. Typically, and in this manifestation, the bias and phototransistor output 801 both become connected to 0V, however it could be any convenient voltage.

Phototransistor bias: Bias is applied to speed-up the response of the phototransistor 804 as well as to provide a source for generating the output signal. As soon as the injection device is activated, the bias may be switched on and remains present until no further doses are expected. Full bias voltage may be developed across the phototransistor 804 at all times, except when a reading is taken. Thus, most of time the phototransistor 804 is being reset in preparation for the next reading. In embodiments, high bias voltages may be unnecessary; the manufacturer tests at 2.0 V. The output signal amplitude is only slightly dependent upon the bias voltage and so a simple low voltage supply is all that is needed. The bias voltage can therefore be generated directly from one of the controller pins.

Sensor IR LED drive: The controller 900 may drive the IR LED 802 with a relatively high current pulse of short duration, typically but not necessarily, a few microseconds long. It may also be driven continuously or with any form of suitably timed waveforms. When operating with pulses, the drive pulse may be applied simultaneously, or a short time after, or before the pin 901 is connected with the ADC input 906, i.e. is setup to sample the phototransistor's 804 output signal.

Sensor IR LED drive duration: The duration of driving the IR LED 802 may be empirically chosen for embodiments. The pulse width of the IR LED drive signal sets the maximum amplitude of the signals. Typically, two sensors or two sensor packages 800 controlled by the controller 900 could have the same drive parameters but that is not essential provided that any introduced timing skew does not adversely affect the encoder function. The drive duration may be set by observations of representative electronics in combination with a reference target, such that the maximum signal would be about 66% (arbitrarily selected) of the full scale of the ADC provided for converting the analogue samples of the output signal of the phototransistor 804 into digital samples. It could be evaluated that at this setting good signal levels may be obtained, well away from saturation, with headroom for tolerances, and high dynamic range whilst using minimal power.

Reading period: This will be defined as the time interval over which the controller 900 has switched its pin 901 from digital drive, for example 0V, to become the ADC input 906 until the pin 901 is set back to digital drive, for example 0V. The reading period will normally be synchronous with the sensor IR LED drive signal but does not have to be simultaneous.

Sensor reading: At, or near, the start of the reading period, the controller 900 may release the digital drive on the pin 901 and set the input 901 as the ADC input 906 in order to read the voltage developed by the sensor, i.e. output at the phototransistor output 801 of the sensor package 800. The input 901 may then become highly capacitive due to its inherent structure. Electric charge from the phototransistor 804 may accumulate on the input capacitance of the ADC input 906 raising the voltage. Since the amount of electric charge from the phototransistor 804 is proportional to the quantity of light that it intercepts, the voltage at the ADC input 906 will be proportional to the integral of that light. After a defined time, the ADC input 906 reading will be made by the controller 900 and then the pin 901 will be connected with the digital drive 908 again, for example driven to 0V. In essence, the pin 901 may be operated in a manner that is similar to a switched capacitor integrator because the 47 kΩ resistor is sufficiently high as to have little impact within the time scale of the embodiment.

ADC configuration: The output from the phototransistor 804 may be digitised via an ADC at a depth of 1 bit or multiple bits. In embodiments, the capacitive ADC input 906 may form part of the function, however, other ADC types with different input structures could be used if suitable external circuitry is included. For example, an implementation could be provided by a comparator (1 bit), or a buffered sample-and-hold circuit could be used with a conventional flash ADC (multi bit), etc.

Power saving: The operation from a power source with low capacity may dictate that the energy used by the encoder can be minimised. To achieve significant energy savings the encoder may be configured to take measurements at particular times, using short pulses on the IR LED drive signal that may create signals that are just adequate for the ADC reading. Between readings, the controller 900 may be able to go into a low power mode and the encoder may only draw thermally induced current from ambient light leakage.

Mitigation of adverse sensor characteristics:
a. A switched capacitor integration may only sample over the period when the signal is expected, thus reducing any influence from non-signal sources.
b. For the rest of the time the maximum bias voltage may be maintained across the sensor in order to sweep away stored or delayed charge within the phototransistor 804.

Multiple sensors: The encoder structure as for example shown in FIGS. 2 and 3 may be quadrature and as such, it may be important that the two sensors 215a and 215b may be sampled closely in time and in some embodiments simultaneously. Significant levels of skew could lead to inaccuracies.

Controlling a sensor of a drug delivery device or of a drug delivery add-on device as described in this disclosure may overcome the normal slow speed response of phototransistors. According to embodiments, a phototransistor is biased nearly all the operation time and its output may be pulled low by a microcontroller output. This output may then be changed to an ADC input just as a light emitting diode (LED) is pulsed.

This may generate a very fast response from the phototransistor which may then be digitised. The ADC may then be disconnected from the pin and the pin may become an output again and pull down the phototransistor output in preparation for the next LED pulse.

As disclosed herein, embodiments may relate to the recording of dialled and/or delivered doses in injection devices, and specific embodiments may relate to application in an injection pen as described in WO2014033195. Furthermore, as disclosed herein, embodiments may be employed with or in an encoder system of an injection device and the embodiments may improve the encoder system's operation.

As disclosed herein, an encoder system may comprise one or more sensors used directly or indirectly to detect a position or positional change. According to embodiments disclosed herein, a rotational position encoder may be employed. The herein disclosed embodiments have several novel features that add significantly to the complexity of an encoder system. For simplicity of explanation, the above detailed description merely describes the components and functions of a single sensor that may be a constituent of the encoder system, and which may be an IR photo-reflective sensor. However, other types of emissive elements and other types of detectors covering the IR spectrum, or any part of the electromagnetic (EM) spectrum, can be used in suitable combination to form a similarly functioning photo-reflective sensor.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short-or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about-4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N—(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present disclosure include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present disclosure, which encompass such modifications and any and all equivalents thereof.

An example drug delivery device may involve a needle-based injection system as described in Table 1 of section 5.2 of ISO 11608-1:2014 (E). As described in ISO 11608-1:2014 (E), needle-based injection systems may be broadly distinguished into multi-dose container systems and single-dose (with partial or full evacuation) container systems. The container may be a replaceable container or an integrated non-replaceable container.

As further described in ISO 11608-1:2014 (E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014 (E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014 (E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

1-15. (canceled)

16. A method of controlling a sensor of a drug delivery device or of a drug delivery add-on device, wherein the sensor comprises a light emitter and a phototransistor as a light receiver, wherein the method comprises:
generating a first drive signal for the light emitter and a second drive signal for the phototransistor, wherein
the second drive signal is generated to bias the phototransistor when the first drive signal is generated to switch off the light emitter, and
the second drive signal is generated to acquire an output signal of the phototransistor when the first drive signal is generated to switch on the light emitter.

17. The method of claim 16, wherein by default the second drive signal is generated to bias the phototransistor.

18. The method of claim 16, wherein generating the first drive signal to switch on the light emitter comprises generating an electric current pulse of a predefined pulse time.

19. The method of claim 18, wherein the predefined pulse time is selected such that the output signal of the phototransistor may reach a predefined value of about 66% of a full scale of an analogue-to-digital converter.

20. The method of claim 18, wherein generating the second drive signal to acquire an output signal of the phototransistor comprises generating a switching signal to connect a signal acquisition input with an output of the phototransistor to receive the output signal of the phototransistor for a predefined acquisition time.

21. The method of claim 20, wherein the predefined acquisition time is longer than the predefined pulse time.

22. The method of claim 16, wherein the second drive signal to acquire an output signal of the phototransistor is generated at approximately a same time of generating the first drive signal to switch on the light emitter or a predefined time delay after generating the first drive signal to switch on the light emitter or a predefined time delay before generating the first drive signal to switch on the light emitter.

23. The method of claim 16, wherein the second drive signal is generated to bias the phototransistor by pulling an output of the phototransistor to a predefined voltage potential.

24. The method of claim 16, comprising:
acquiring the output signal of the phototransistor by sampling and converting the output signal into a digital signal.

25. A device for controlling a sensor of a drug delivery device or a drug delivery add-on device, wherein the sensor comprises a light emitter and a phototransistor as a light receiver, wherein the device is configured to perform operations comprising:
generating a first drive signal for the light emitter and a second drive signal for the phototransistor, wherein
the second drive signal is generated to bias the phototransistor when the first drive signal is generated to switch off the light emitter, and
the second drive signal is generated to acquire an output signal of the phototransistor when he first drive signal is generated to switch on the light emitter.

26. The device of claim 25, comprising a controller configured to generate the first drive signal for the light emitter and the second drive signal for the phototransistor.

27. The device of claim 26, comprising an input for connecting to an output of the phototransistor to receive the output signal of the phototransistor, wherein the input can be controller-internally switched between a predefined voltage potential, and an input of an analogue-to-digital converter comprised by the controller.

28. The device of claim 27, wherein the input of the analogue-to-digital converter comprises an input capacitance for receiving an electric charge from the phototransistor and is selected to obtain a rising time of an input voltage of the analogue-to-digital converter being lower than a predefined rising time.

29. A sensor for a drug delivery device or for a drug delivery add-on device, wherein the sensor comprises:
at least one sensor unit comprising a light emitter configured to emit light in a first frequency range and a phototransistor configured to detect received light in a second frequency range, wherein the second frequency range comprises the first frequency range, and
a device configured to control the at least one sensor unit, wherein the device is configured to perform operations comprising:
generating a first drive signal for the light emitter and a second drive signal for the phototransistor, wherein
the second drive signal is generated to bias the phototransistor when the first drive signal is generated to switch off the light emitter, and
the second drive signal is generated to acquire an output signal of the phototransistor when the first drive signal is generated to switch on the light emitter.

30. The sensor of claim 29, wherein the light emitted in the first frequency range by the light emitter comprises a first peak wavelength and a first full-width half-maximum.

31. The sensor of claim 30, wherein a light detection spectrum of the phototransistor comprises a second peak wavelength and a second full-width half-maximum, wherein the first peak wavelength and the first full-width half-maximum are selected such that the light detection spectrum of emitted light is comprised by the light detection spectrum and light emitted from the light emitter and received by the phototransistor generates a signal level of the output signal of the phototransistor sufficient for further processing by the device.

32. The sensor of claim 31, wherein the first peak wavelength is about 936 nm and the first full-width half-maximum is about 59 nm, and wherein the second peak wavelength is about 872 nm and the second full-width half-maximum 276 nm.

33. The sensor of claim 29, wherein the phototransistor is connected to a load of about 47 kohm.

34. The sensor of claim 29, wherein the drug delivery device comprises an injection pen comprising:
a body for holding a drug container,
a dosage selection mechanism for selecting a drug dosage to be delivered, which comprises an optical encoder system for detecting a selected and/or delivered drug dosage, and
wherein the sensor is arranged to detect movement of a part of the optical encoder system upon drug dosage selection and/or delivery based on the detection of reflections of emitted light from a moving part of the optical encoder with the phototransistor.

35. The sensor of claim 29, wherein the optical encoder system comprises a light source and a light detector.
